(19) United States
(12) Patent Application Publication
(10) Pub. No.: US 2005/0084862 A1
(43) Pub. Date: Apr. 21, 2005

(54) Sample preparation for the detection of infectious agents

(76) Inventors: Helen Lee, Cambridge (GB); Ling Huang, Cambridge (GB); Elpidio Casar, San Mateo (CA); Nigel Derek Buttress, San Mateo, CA (US)

Correspondence Address: PILLSBURY WINTHROP LLP, ATTENTION: DOCKETING DEPARTMENT, 11682 EL CAMINO REAL, SUITE 200, SAN DIEGO, CA, 92130, US

(21) Appl. No.: 10/500,167

(22) PCT Filed: Dec. 24, 2002
(86) PCT No.: PCT/GB02/05923

(30) Foreign Application Priority Data
Dec. 24, 2001 (GB) 0130947.5

(51) Int. Cl. C12Q 1/68

(52) U.S. Cl. 435/6

(57) ABSTRACT

Methods for improving the quality of sub-optimal patient samples for detection of infectious agents are described. In particular, endocervical fluid samples or vaginal fluid samples are treated with DNase to improve the reliability of detection of infectious agents. Kits for carrying out the methods are also described.

The present invention relates to means for improving disease detection, improved methods for testing for the presence of disease-indicating moieties in a test sample and kits for carrying out such methods.

### BACKGROUND

It is desirable to perform disease detection tests on patient samples in a manner which provides a reliable disease-specific result and in a manner which does not provide a high level of false positive or false negative results. Disease detection may be by detection of a particular analyte in a sample, for example by detection of a particular antigen or antigenic fragment, antibody or antibody fragment, or nucleic acid sequence. It is preferable if the patient sample requires the minimum amount of preparation and handling prior to testing. It is also preferable if the patient sample can be obtained in a completely or relatively non-invasive manner.

One conventional method for testing for the presence of an analyte in a test solution comprises capturing the analyte on a dipstick and detecting for the presence of the analyte on the dipstick The dipstick has a contact end for contacting the test solution and a capture zone remote from the contact end. The test solution is caused to be drawn from the contact end to the capture zone where any analyte present in the test solution should be captured and an appropriate display provided as a readout. Known types of dipstick use antibody-antigen reactions to detect an analyte. The analyte may be directly indicative of an infection by an infectious agent or alternatively may be indirectly indicative of a disease state.

Under controlled test conditions (particularly under laboratory test conditions) it may be possible to carry out detection tests on optimized samples. Such samples may be optimized both in terms of the “loading” of analyte to be detected (ie the organism load) and also in the quality and quantity of the test sample in which the analyte is presented for detection.

In contrast, in many clinical situations, patient samples are collected in sub-optimal conditions. Furthermore, patient samples may themselves be sub-optimal: for example they may have widely varying loadings between samples and/or may be mixed with and contaminated by many other materials which may interfere with the test procedure. In addition, these materials may vary widely between individual samples in terms of their quantity and/or composition. The individual variation may depend on the physiological condition, health status and/or dietary habit.

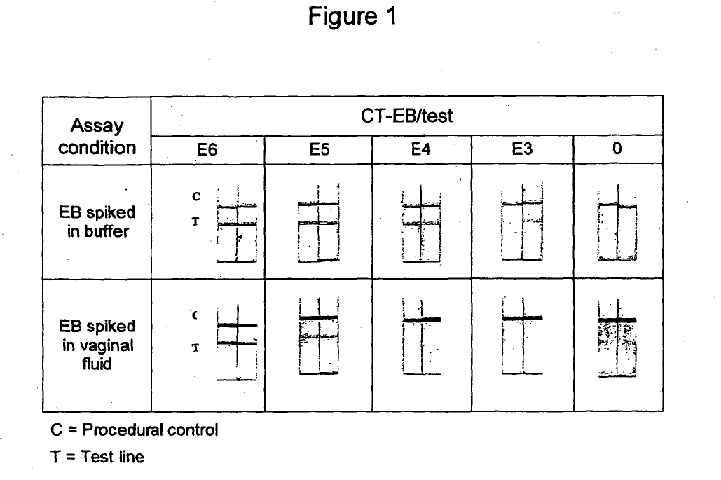

As a consequence, some tests which might be proposed to be effective in buffers may give unreliable results or may fail to work at all when applied to real patient samples. An example of this is the detection of Chlamydia trachomatis (CT), the cause of infertility and pelvic inflammatory disease in women. FIG. 1 shows the detection level when the elementary bodies (EB) of CT is spiked in a buffer compared to that spiked in vaginal fluid. It can be seen that there is a decrease of the test signal by approximately 100 fold due to inhibitory substances present in the vaginal sample.

Manual or automated tests such as Enzyme immunoassays (EIA) rely on sequential steps of incubation with reagents and washing to improve the test sensitivity and reduce the inhibitory effect of sample on the test results. However, in the case of dipstick assays, the biological sample itself serves as a liquid that dissolve the dried reagents impregnated in the dipstick Because there is no incubation or wash step and because the reaction is rapid, the effect of the quality of the sample is particularly important.

Arko et al (Journal of Clinical Microbiology (1979), 9, 517-519) discloses nuclease enhancement of specific cell agglutination in a slide agglutination test (SAT) for detection of Neisseria gonorrhoeae. However, the test was performed on laboratory cultures (bacterial cells grown on agar suspended in PBS buffer containing DNase at 1 mg per ml). There is no disclosure of how to improve testing of patient samples (in particular of endocervical fluid samples or vaginal fluid samples), nor any disclosure that nuclease treatment may enhance tests other than the SAT. Indeed, nuclease treatment did not improve fluorescent staining of gonococci in a fluorescent antibody test.

Tarkowski et al (Molecular Diagnosis (2001), 6(2), 125-130) describes improved detection of viral RNA isolated from liquid-based cytology samples. Total nucleic acid (TNA) was extracted from cell lines grown in the laboratory (either from fresh cells or from cells fixed in liquid-based cytology media). The extracted TNA was then treated with DNase-I to allow analysis of the RNA in the samples by RT-PCR. However, there is no disclosure of testing of clinical samples, nor of how these may be treated to improve the sensitivity, specificity, or reliability of the tests.

Hence, there is a desire to provide improved disease detection tests which are capable of performing to an acceptable level of sensitivity, specificity, reliability and ease of use on patient samples irrespective of the “quality” of the sample.

### BRIEF DESCRIPTION OF THE INVENTION

According to the present invention there is provided a means for improving the “quality” of sub-optimal patient samples for detection of infectious agents. The invention provides several steps which can be taken alone or in combination which result in improved test performance by sub-optimal patient samples.

In a preferred aspect, the invention provides a method for treatment of a human patient sample for carrying out a diagnostic method on the sample for detection of an infectious agent, wherein the sample is an endocervical fluid sample or a vaginal fluid sample, which includes the step of carrying out the diagnostic method in the presence of DNase. Preferably the endocervical fluid sample or the vaginal fluid sample is treated with DNase.

It will be appreciated that the invention does not require a prior knowledge of the quality of the patient sample: samples which are of poor quality will be improved and samples which are already of high quality might be unaffected or marginally improved. The overall effect is that a highly reliable test result can be obtained on patient samples irrespective of the original quality without the need for quality testing.

The invention will now be described in detail with regard to testing for the sexually transmitted disease caused by Chlamydia trachomatis by the analysis of self-collected vaginal swabs as the sample type. However, the invention is not to be construed as being limited to the preferred examples, and is only defined in scope by the claims.

The Importance of Chlamydia as a Disease

CT is the most common sexually transmitted pathogen in western countries. CT serotypes D-K have a worldwide distribution and are the cause of genital infection and associated ocular and respiratory infections. Chlamydia infection may lead to serious complications and sequelae: for example Pelvic Inflammatory Disease (PID), ectopic pregnancy, infertility in women, and perinatal and congenital infections in babies born from infected mothers.

CT infections often remain undiagnosed because many infected women have mild, non-specific symptoms or are asymptomatic. Screening for CT infection is a critical component of control efforts. Diagnosis of CT infection in women using EIA or rapid tests currently requires a pelvic examination. Only amplified nucleic acid tests such as Ligase Chain Reaction (LCR) or Polymerase Chain Reaction (PCR) are capable of testing urine samples. Testing with LCR or PCR is complex, requires expensive instruments and the test result is not usually available until a week after the taking of the sample. The use of non-invasive samples (i.e. urine or self-collected vaginal swabs) with rapid tests could allow testing of more women including those who are either deterred by a pelvic examination or do not have access to one (i.e. due to lack of access to a trained medic or physician).

LCR testing of paired urine and vaginal swab samples from women attending a genitourinary clinic have shown that there are lower counts of CT organism in urine compared to vaginal fluids. Therefore, the use of self-collected vaginal sample has the advantage of being a non-invasive sample type that is simple to collect. Due to its higher organism load the use of this sample would also improve the test sensitivity compared to urine.

Current protocols for urine testing require a dilution step with water before centrifugation to reduce the inhibitory effect of urine to the test. In addition, the use of urine as a sample requires a concentration step, which not only adds to the cost but to the processing time. One example of this is the Clearview Chlamydia ME (Unipath LTD, Bedford, England) test where 10ml of urine specimen is diluted with an equal volume of distilled or deionised water before centrifugation at 3000 g for 15 minutes.

Although no current test has a product claim for vaginal swab sample, we have found that vaginal swab samples are inhibitory to current antibody-based or amplified DNA Chlamydia tests. These antibody-based tests include Clearview Chlamydia MF, QuickVue Chlamydia (Quidel Corp, CA, USA), and Chlamydia OIA (Biostar, CO, USA).

The Clearview Chlamydia MF test is a lateral flow immunoassay intended for detection of Chlamydia trachomatis antigen in either female endocervical swab specimens or male urine specimens. The male urine specimens require dilution and centrifugation step as mentioned above while the swab specimens do not. Extraction involves addition of extraction reagent, heating at 80° C. for 10 minutes, and allowing the sample to cool for at least 5 minutes. Test procedure involves applying 5 drops of the sample extract to the sample window of the test unit (immunochromatographic test strip) and reading the result after 15 minutes.

The QuickVue Chlamydia test is a lateral flow imununoassay intended for detection of Chlamydia antigen from endocervical swab and cytology brush specimens. To perform the test, an endocervical clinical specimen is obtained and placed into a tube containing the extraction solution; after 2 minutes, neutralization solution is added to the tube. After extraction and neutralization, 3 drops of extracted sample is added to the sample well of the test cassette and the results read after 10 minutes.

The Chlamydia OIA is an optical immunoassay intended for detection of Chlamydia antigen from female endocervical swabs and neonatal conjunctival specimens. The test involves the extraction of Chlamydia Lipopolysaccharide (LPS) from the specimen using two specimen extraction reagents and a neuralization reagent, and the subsequent use of Optical Immunoassay technology for the qualitative detection of the antigen. The detection or testing stage involves five steps: application of the sample extract to the test device (5-min incubation), addition of the conjugate (5-min incubation), washing, addition of the substrate (5-min incubation), and a final wash. Test results are evaluated under a bright light source with light reflected off the test surface.

We have developed a rapid immunochromatographic strip test for the detection of Chlamydia LPS antigen using self-collected vaginal swabs. To perform the test, the swab specimen is placed into a tube containing the extraction solution. The sample extract is neutralized, and an aliquot is applied to an immunochromatographic test strip (i.e. a “dipstick”). The test results are read after 15-25 minutes.

Sample Preparation

An inhibitory effect of vaginal fluid on the assay sensitivity was observed when known amounts of EB's were spiked into vaginal swabs (FIG. 1). The signals generated in the present of vaginal fluid showed a reduction of approximately 100 fold compared to buffer. There are at least two aspects to the inhibitory phenomenon observed with vaginal swab specimens: direct inhibition of antibody-antigen interaction and indirect inhibition of the test by preventing proper mixing of reagents and the reduction or inhibition of liquid flow.

The vaginal sample contains components that directly inhibit the interaction between antibodies and their target antigen (i.e. LPS and anti-LPS antibodies). This inhibition may be through physically blocking the antibody and antigen from coming together, sequestration of the LPS target, or modification of charges on the antibody molecule adversely affecting its affinity. The inhibitory effect varies widely between individuals and within the same individual during different periods of the menstrual cycle.

Inhibition of proper mixing and liquid flow is related to the inherent viscosity of vaginal fluid. Two of the main contributors to this viscosity are mucin levels and amounts of DNA in the sample. The high mucin level in some samples contributes to the viscosity and large amounts of DNA tend to form a gel-like matrix thus physically clog the membrane and prevent or reduce fluid flow.

Vaginal fluids are highly variable from individual to individual and during different periods within a woman's monthly menstrual cycle. The presence of other things like sperm, excessive bacterial growth, yeast infection, vaginal douches, and lubricants may also contribute to this variability.

### GENERAL EXAMPLE:

### Vaginal Swab Samples

Sample collection

1. Use either a polyurethane or polyester (e.g. Dacron) swab (on a polystyrene or polypropylene plastic shaft) to collect the vaginal sample. Tampons and sanitary napkins may also be used for sample collection.
2. The swab should be inserted preferably 6 cm (3 cm to 9 cm) deep from the opening of the vagina and rotated several times for at least 10 seconds before removal.
3. The sample can either be stored dry or in sample collection buffer at 2-8° C. for 2-4 days before testing.
   Sample preparation
   1. Use of DNase to degrade nucleic acids present

Some vaginal swab samples contain large amounts of DNA which forms a gel-like matrix that tend to retain fluid, clog the nitrocellulose membrane and inhibit the migration of reagents, and results in a total failure of the test. Digestion of the DNA with DNase prevents the above from happening. DNase is effective when added at more than 0.5 jig/ml or 1.5 units of activity per ml, for example 0.5-100 μg/ml or 1.5-300 units of activity per ml. The required amount of DNase ultimately depends on the amount of DNA in the sample and the length of time given for the enzyme to act.

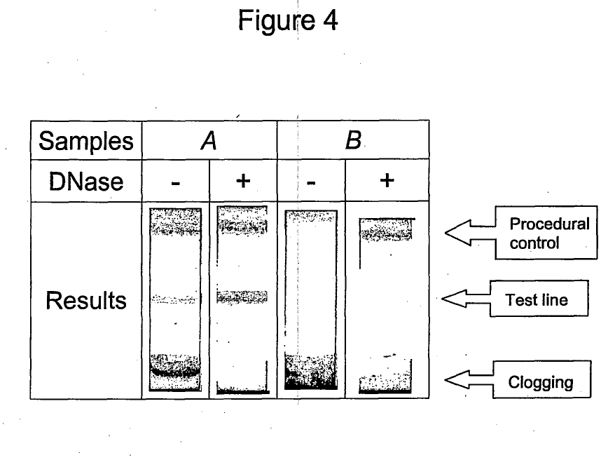

FIG. 4 shows the beneficial effects of DNase treatment on the dipstick assay. In the vaginal fluid of an individual infected with Chlamydia, treatment with DNase prevented clogging at the bottom of the strip and allowed a stronger positive signal to develop at the test line. In the Chlamydia negative vaginal swab sample B, clogging at the bottom of the strip was prevented by DNase treatment, enabling the procedural control signal to develop.

Neuraminidase and lysozyme were also tested along with DNase for their effect on the inhibitory property of vaginal fluid. Unlike DNase however, neither enzyme consistently affected the inhibitory phenomenon of vaginal fluid. Neuraminidase was tested from 3 to 200 units of activity per ml of vaginal sample extract while lysozyme was tested at 830 to 100,000 units per ml of vaginal sample extract.

2. Use of n-dodecyl maltoside as a surfactant

The non-ionic alkyl glucosides, particularly n-dodecyl maltoside, are the most effective surfactants for extracting CT LPS from vaginal samples and making it available for antibody detection. N-dodecyl maltoside works best at concentrations of 0.01% to 0.04% w/v, preferably 0.015% to 0.03% w/v.

Table 1 shows that when vaginal swabs were taken from different individuals (A to G), spiked with 20,000 Chlamydia EB per swab, the addition of n-dodecyl maltoside to the extraction buffer gave the best signal results.

3. Use of PVA or PVP

Polyvinyl alcohol (PVA) is an effective blocking agent when using vaginal swab samples. PVA is thought to coat the nitrocellulose membrane fibres, effectively blocking it from binding other reagents. This allows more reagent to be available for reaction at the capture line. It also results in a cleaner background for the test. In addition, it enhances the sensitivity of the dipstick test either by acting as an LPS carrier or by enhancing the formation of LPS micelles. PVA works best at concentrations of 0.01% to 0.5% w/v. Polyvinyl pyrrolidone (PVP) also enhances the sensitivity of the test through a mechanism similar to that of PVA and works best at 0.2% to 2% w/v.

Table 2 shows that when vaginal swabs were taken from different individuals (A to G), spiked with 20,000 Chlamydia EB per swab, and tested with or without PVA in the extraction solution, the signals were stronger in those extracted in the presence of PVA.

4. Use of H₂O₂ to oxidize inhibitory substances

The addition of H₂O₂ is able to neutralize some of the inhibitory effects of vaginal samples. This effect may be associated with H₂O₂ as an oxidizing agent and therefore neutralizing some of the inhibitory substance(s) by oxidation. The optimum concentration of H₂O₂ is between 0.5% to 3% w/v. It becomes less effective below this range and begins to adversely affect the test above this range.

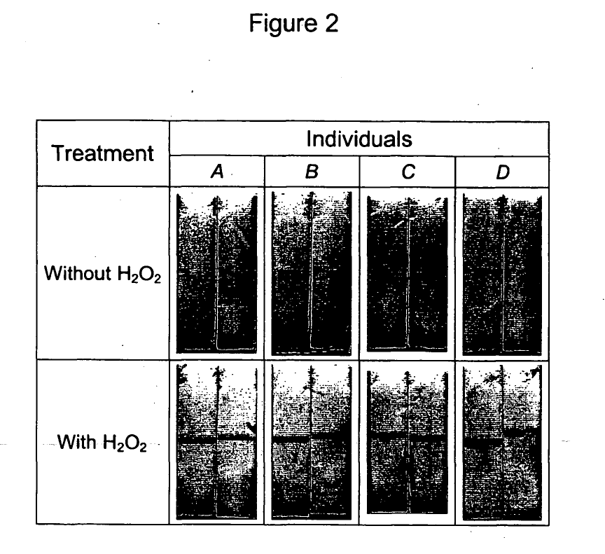

FIG. 2 shows the effect of H₂O₂ on the signal of a CT dipstick test in different individuals. Vaginal swabs were collected from four different individuals (A to D) and spiked with 90,000 Chlamidia EB per swab The extraction step was carried out with or without the addition of 1% H₂O₂ . It can be seen that in samples not treated with H₂O₂ , no positive signal was visible whereas in those treated with H₂O₂ all yielded strong positive signals.

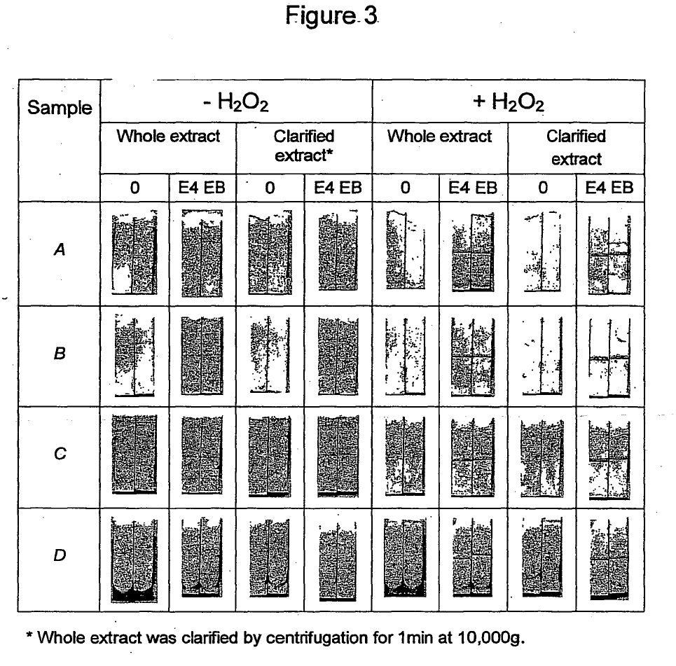

As demonstrated by FIG. 3, the inhibitory substance(s) exist mainly in the soluble fraction of the processed samples. Vaginal swabs taken from individuals A-D were spiked with 80,000 EB's per swab. After extraction of LPS, half of the samples were further clarified by centrifugation to remove particular matters. Both centrifuged and non-centrifuged samples were tested for the presence of LPS with or without H₂O₂ treatment. It can be seen that a marked improvement of signal strength was observed in all samples with H₂O₂ treatment.

General example of a sample preparation procedure:

A self-collected vaginal swab specimen is first obtained and then treated in the following manner. (Note that if a tampon or sanitary napkin is used to collect the specimen, the amounts of reagents used will have to be adjusted accordingly.)
1. Add 400 μl reagent A to the swab to disrupt EB and extract LPS. Reagent A contains 100 to 300 mM NaOH. The swab should be allowed to incubate in reagent A for no longer than 5 minutes.
2. Add 300 μl reagent B to decrease the pH, provide a protein and polymer blocker, and form LPS micelles. Reagent B is a 0.5M Tris buffer, pH8.5 containing 100 mM NaCl, 130-400 mM HCl and 1%-4% protein such as bovine serum albumin, 0.03%-1.3% polyvinyl alcohol and 0.03%-0.1% n-dodecylmaltoside. The swab should not be allowed to incubate for more than 5 minutes.
3. Add 100 μl reagent C to oxidize inhibitory substances. Reagent C is 6% H₂O₂. The sample should not be allowed to incubate for more than 2 minutes.
4. Apply the sample to the test strip in the presence of more than 0.5 μg/ml or 1.5 units of activity per ml, for example 0.5-100 μg/ml or 1.5-300 units of activity per ml DNase.
   References

Smith K, Harrington K, Wingood G, Oh MK, Hook EW, DiClemente RJ. 2001. Self-obtained vaginal swabs for diagnosis of treatable sexually transmitted diseases in adolescent girls. Arch Pediatr Adolesc Med 155:676-679.

TABLE 1

| | Individuals | | | | | | |
|---|---|---|---|---|---|---|---|
| Surfactants | A | B | C | D | E | F | G |
| n-Dodecyl maltoside | 1.5 | 2 | 2 | 1.5 | 2 | 2 | 2 |
| n-octyl glycopyranoside | 1 | 1.5 | 0.5 | 1 | 1.5 | 2 | 2 |
| Tween 20 | 2 | 0.5 | 1 | 0.5 | 1 | 1.5 | 2 |
| Chapso | 0.5 | 0.5 | 0.5 | 0.5 | 1 | 1 | 1 |
| Cholate | 0.5 | 0.5 | 1 | 0.5 | 0.5 | 1 | 1 |

The signals are on a scale of 1 to 5 with 5 being the strongest signal.

TABLE 2

| | Individuals | | | | | | |
|---|---|---|---|---|---|---|---|
| Treatment | A | B | C | D | E | F | G |
| Without PVA | 1 | 1.5 | 1 | 1.5 | 1 | 0.5 | 0.5 |
| With PVA | 2 | 2.5 | 2 | 2.5 | 2 | 2.5 | 1.5 |

The signals are on a scale of 1 to 5 with 5 being the strongest signal.

1. A method for treatment of a human patient sample for carrying out a diagnostic method on the sample for detection of an infectious agent, wherein the sample is an endocervical fluid sample or a vaginal fluid sample, which includes the step of carrying out the diagnostic method in the presence of DNase.

2. A method according to claim 1 wherein the DNase is present in an amount of more than 0.5 μg/ml, preferably 0.5 to 100 μg/ml.

3. A method according to claim 1 wherein the DNase is present in an amount of more than 1.5 units of activity per ml, preferably 1.5 to 300 units activity per ml.

4. A method according to any of claims 1 to 3 which additionally includes a method for preparation of a human patient sample prior to carrying out a diagnostic method on the sample for detection of an infectious agent, which preparative method includes the step of treating the sample with an oxidizing agent.

5. A method according to claim 4 wherein the oxidizing agent is hydrogen peroxide (H₂O₂).

6. A method according to claim 5 using a working concentration of hydrogen peroxide of 0.5% to 3% w/v.

7. A method according to any of claims 1 to 3 or 4 to 6 which additionally includes the step of treating the sample with a non-ionic alkyl glucoside surfactant.

8. A method according to claim 7 wherein the surfactant is n-dodecyl maltoside.

9. A method according to claim 8 wherein the n-dodecyl maltoside is present at a working concentration of 0.01% to 0.04% w/v, preferably 0.015% to 0.03%.

10. A method according to any of claims 1 to 3 or 4 to 6 or 7 to 9 which additionally includes the step of treating the sample either or both of PVA and PVP.

11. A method according to claim 10 wherein wherein the sample is treated with PVA, preferably having an average molecular weight between 20 and 25 kDa and at a working concentration of between 0.01 and 0.5% w/v.

12. A method according to claim 10 wherein the sample is treated with PVP at a working concentration between 0.2% and 2% w/v.

13. A method according to any of claims 1 to 3, which additionally comprises a method step according to any of claims 10 to 12 and a method step according to any of claims 7 to 9 and a method step according to any of claims 4 to 6.

14. A method according to any of claims 1 to 13 wherein the human patient sample is obtained as a self-collected vaginal swab sample.

15. A method according to any of claims 1 to 13 wherein the method is for detection of Chlamydia trachomatis.

16. A method according to any of claims 1 to 13 wherein the patient sample is a self-collected vaginal swab sample and the method is for detection of Chlamydia trachomatis.

17. A method according to any preceding claim wherein the method is a dipstick test method.

18. A kit comprising:
a dipstick test apparatus for carrying out a specific infectious agent detection test;
reagents required for said apparatus in order to carry out said specific detection tests;
a DNase reagent for carrying out the method of any of claims 1 to 3.

19. A kit according to claim 18 additionally comprising:
an oxidizing agent reagent for carrying out the method of any of claims 4 to 6.

20. A kit according to claim 18 additionally comprising:
a non-ionic alkyl glucoside reagent for carrying out the method of any of claims 7 to 9.

21. A kit according to claim 18 additionally comprising:
a reagent which is PVA and/or PVP for carrying out the method of any of claims 10 to 12.

22. A kit according to claim 18 additionally comprising:
a non-ionic alkyl glucoside surfactant reagent as defined in any of claims 4 to 6 and a PVA and/or PVP reagent as defined in any of claims 7 to 9 for carrying out the method of any of claims 1 to 15.
